(19) United States
(12) Patent Application Publication
(10) Pub. No.: US 2026/0128154 A1
(43) Pub. Date: May 7, 2026

(54) SYSTEM AND METHOD FOR GENERATING PERSONALISED DIETARY RECOMMENDATION

(71) Applicant: PIKKY TECHNOLOGIES INCORPORATED, Fremont, CA (US)

(72) Inventor: Mohtashim Arbaab QURESHI, Fremont, CA (US)

(21) Appl. No.: 18/686,191

(22) PCT Filed: Mar. 14, 2022
(86) PCT No.: PCT/IB2022/052284

§ 371 Date: Feb. 23, 2024

(30) Foreign Application Priority Data
Aug. 23, 2021 (IN) 202111038133

(51) Int. Cl. G16H 20/60; G06F 16/9038

(52) U.S. Cl. CPC G16H 20/60; G06F 16/9038

(57) ABSTRACT

The present invention relates to a system and method for generating dietary recommendation information for a user, and more particularly to a computer implemented method and system which generates and presents personalised dietary recommendation information to a user. The system and method as per the present invention constructs profile of a plurality of dietary items which includes ingredient profile, sensory profile and geographical profile, before constructing and mapping the profile of a user against the profile of a plurality of dietary items to determine confidentiality scores and generate dietary recommendations to the user.

### TECHNICAL FIELD

The present invention relates to a system and method for generating dietary recommendation information for a user, and more particularly to a computer implemented method and system which generates and presents personalised dietary recommendation information to a user.

### BACKGROUND OF INVENTION

Food is an essential part of daily routine for humans, and variety in food is commonly craved. People, however, generally struggle while choosing what food to eat at different time during the day. This is primarily because of indecisiveness which stems from the availability of overwhelmingly large number of ever increasing options to select from. A lot of energy, time and effort goes into the decisions and choices we make on a daily basis in terms of what to eat. Confusion & indecisiveness prevails in meal planning, while cooking as well as ordering. This is primarily due to excessive and unfiltered information.

The situation gets complicated and aggravated when people travel to different places and countries, and are not very familiar with the local cuisine of the new place. Even the ingredients, preparation and serving of universal dishes, such as pasta, pizza, etc. varies significantly from one place to another, which results in either more indecisiveness or often disappointment with the flavours and taste.

Furthermore, for people having allergies or having medical conditions it becomes even more important and difficult to find the right dietary option or food which is consistent with the allergies and/or medical conditions and also good in taste and sensory experience.

Also, the dietary habits and patterns of individuals varies significantly and depend upon several internal factors such as personal likes and dislikes, allergies, etc. as well as external factors such as time of the day, occasion, mood, cost, etc. whether consciously or sub-consciously.

While there are certain platforms which help order the food, but what is lacking is personalization. It is, therefore, desirable to have a system and method which could generate and present personalized recommendation information for dietary items. Accordingly, it is desirable to have a recommendation system that understands and analyses human conscious and subconscious behaviour patterns and sensory inclinations towards aiding in their decision-making process.

### OBJECTS OF THE INVENTION

The main object of the present invention is to provide a computer implemented method and system for generating personalized dietary recommendation information for a user.

Another object of the present invention is to provide a computer implemented method and system for generating personalized dietary recommendation information for a user wherein the ingredients of the recommended dietary items are as per the user's dietary preferences such as vegetarian, pescatarian, kosher, halal, etc.

Yet another object of the present invention is to provide a computer implemented method and system for generating personalized dietary recommendation information for a user wherein the ingredients of the recommended dietary items takes into account any allergies and medical conditions such as shellfish allergy, peanut allergy, gluten or dairy allergy, diabetic food, etc.

Still another object of the present invention is to provide a computer implemented method and system for generating personalized dietary recommendation information for a user wherein the recommendation takes into account user's cognitive behaviour and patterns such as moods, emotions, health goals, allergies, medical conditions, food habits, etc.

Yet another object of the present invention is to provide a computer implemented method and system for generating personalized dietary recommendation information for a user wherein the recommendation takes into account user's external factors such as user's current location, weather conditions, time of the day, occasion, space, ambience, etc.

Another primary object of the present invention is to provide a computer implemented method and system for generating personalized dietary recommendation information for a user wherein the recommendation takes into account user's sensory attributes and preference in terms of taste, aroma, texture, and visuals of the dietary items.

Yet another object of the present invention is to provide a computer implemented method and system for generating personalized dietary recommendation information for a user wherein the recommendation takes into account user's comfort food and willingness to try new dietary items outside the comfort food options.

Still another object of the present invention is to provide a computer implemented method and system for generating personalized dietary recommendation information for a user wherein the recommendation information could be in the form of a dietary item, recipe of a dietary item, a cuisine, or a restaurant.

The other objects, preferred embodiments and advantages of the present invention will become more apparent from the following detailed description of the present invention when read in conjunction with the accompanying claims, examples, figures and tables, which are not intended to limit scope of the present invention in any manner.

### SUMMARY OF THE INVENTION

Accordingly, the present invention and the embodiments encompassed herein provide a computer implemented method and system for personalized dietary recommendations for users.

As per one of the most general embodiments of the present invention is provided a computer implemented method for generating custom dietary recommendation information for a user, the method comprising: (a) Compiling a database of plurality of dietary items; (b) Constructing a profile for each of the dietary item in the database, wherein the profile of each of the dietary item comprises: (i) an ingredient profile of the dietary item; (ii) a sensory profile of the dietary item, wherein the sensory profile comprises: a visual profile comprising visual tags depicting visual attributes of the dietary item; a taste profile comprising taste tags depicting taste attributes of the dietary item; an aroma profile comprising aroma tags depicting aroma attributes of the dietary item; a texture profile comprising texture tags depicting texture attributes of the dietary item; and (iii) A geographical profile of the dietary item, identifying at least one geographical territory associated with the dietary item; and (c) Acquiring information and preferences from the user to construct user profile wherein the user profile comprises at least: one preferred ingredient or one preferred dietary item or one preferred geographical location; (d) Mapping the user profile against the profile of a plurality of dietary items in the database; (e) Constructing a confidence score for each of the mapped dietary items; (f) Creating a set of dietary items having confidence score within a range, wherein the said range is calibrated with the user's profile; (g) Generating recommendation information for the user wherein the recommendation information comprises at least one dietary item selected from the set of dietary items having the confidence score within the range; and (h) Presenting the recommendation information to the user.

The present invention also provides a system, wherein the system comprises: one or more computer-readable memory devices storing data and instructions; one or more input-output devices with graphic user interfaces; one or more data processing apparatuses configured to interact with one or more memory devices and input-output devices, wherein upon execution of the instructions stored in the memory devices the system perform operations including: (a) Compiling a database of plurality of dietary items and storing it in one or more computer-readable memory devices; (b) Constructing a profile for each of the dietary item in the database, wherein the profile of each of the dietary item comprises: (i) an ingredient profile of the dietary item; (ii) a sensory profile of the dietary item, wherein the sensory profile comprises: a visual profile comprising visual tags depicting visual attributes of the dietary item; a taste profile comprising taste tags depicting taste attributes of the dietary item; an aroma profile comprising aroma tags depicting aroma attributes of the dietary item; a texture profile comprising texture tags depicting texture attributes of the dietary item; (iii) A geographical profile of the dietary item, identifying at least one geographical territory associated with the dietary item; (c) Acquiring, through an input-output device with a graphic user interface, information and preferences from the user to construct user profile wherein the user profile comprises at least: one preferred ingredient or one preferred dietary item or one preferred geographical location; (d) Mapping the user profile against the profile of a plurality of dietary items in the database by the processing apparatus; (e) Constructing a confidence score for each of the mapped dietary items; (f) Creating a set of dietary items having confidence score within a range, wherein the said range is calibrated with the user's profile; (g) Generating recommendation information for the user wherein the recommendation information comprises at least one dietary item selected from the set of dietary items having the confidence score within the range; and (h) Presenting the recommendation information to the user through the input-output device with a graphic user interface.

The present invention further provides a system for generating and presenting dietary recommendation to a user, the system comprising: (a) a computer readable storage device storing a database of a plurality of dietary items; (b) a device configured to generate and store profile of each dietary items in the database, wherein the profile of each dietary item comprises: (i) an ingredient profile of the dietary item; (ii) a sensory profile of the dietary item, wherein the sensory profile comprises: a visual profile comprising visual tags depicting visual attributes of the dietary item; a taste profile comprising taste tags depicting taste attributes of the dietary item; an aroma profile comprising aroma tags depicting aroma attributes of the dietary item; a texture profile comprising texture tags depicting texture attributes of the dietary item; and (iii) A geographical profile of the dietary item, identifying at least one geographical territory associated with the dietary item; (c) a graphic user interface (GUI) configured to interact with the user to receive the user's preferences and shared recommendations; (d) a device configured to generate profile of the user based on the preferences shared by the user, wherein the use profile comprises at least: one preferred ingredient or one preferred dietary item or one preferred geographical location; (e) a mapping device configured to map the user profile against the profile of a plurality of dietary items in the database; and (f) a processor device coupled with the mapping device to constructing a confidence score for each of the mapped dietary items and to segregate the information of dietary items having confidence score within a range, wherein the said range is calibrated with the user's profile; (g) a device to generate recommendation information for the user wherein the recommendation information comprises at least one dietary item selected from the set of dietary items having the confidence score within the range; and (h) an output device to transmit the generated recommendation to user's graphic user interface and to receive the user's response.

### BRIEF DESCRIPTION OF THE DRAWINGS

Other features, objects, and advantages of present disclosure will become more apparent by reading the detailed description of non-limiting embodiments made with reference to the following drawings. The accompanying drawings, which are incorporated in and constitute a part of this specification, are only for illustration of certain examples and embodiments of the present invention and the subject matter disclosed herein. Together with the description, these drawings help explain some of the principles associated with the implementation and various aspects of the present invention.

FIG. 1 illustrates a conceptual block diagram of an environment in which the present invention can operate.

FIG. 2 is a diagrammatic representation illustrating the system and method for constructing the profile of a dietary item/cuisine.

FIG. 3 is a flowchart illustrating the components of a sensory profile in accordance with one of the embodiments of the invention.

FIGS. 4A and 4B are a diagrammatic representation depicting different Olfactory/Aroma Tags (400) in an Olfactory/Aroma Profile of a dietary item, according to an embodiment of the invention as disclosed herein. The tags and the scheme of categorisation as disclosed in the diagram are merely illustrative, and are not meant to be exhaustive or to limit the scope of invention in any manner.

FIG. 5 is a flowchart representation illustrating a systematic method of constructing an Aroma/Olfactory Profile (502) of a dietary item, according to an embodiment of the invention as disclosed herein.

FIGS. 6A and 6B are diagrammatic representations depicting different Gustatory/Taste Tags (600) in the Gustatory/Taste Profile of a dietary item, according to an embodiment of the invention as disclosed herein. The tags and the scheme of categorisation as disclosed in the diagram are merely illustrative, and are not meant to be exhaustive or to limit the scope of invention in any manner.

FIG. 7 is a flowchart representation illustrating a systematic method of constructing an Gustatory/Taste Profile (702), of a dietary item, according to an embodiment of the invention as disclosed herein.

FIGS. 8A and 8B are a diagrammatic representation illustrating different Visual Tags (800) of Visual Profile, of a dietary item, according to an embodiment of the invention as disclosed herein. The tags and the scheme of categorisation as disclosed in the diagram are merely illustrative, and are not meant to be exhaustive or to limit the scope of invention in any manner.

FIG. 9 is a flowchart illustrating a systematic method of constructing a Visual Profile (902) of a dietary item, according to an embodiment of the invention as disclosed herein.

FIG. 10 is a diagrammatic representation illustrating different Texture Tags (1010) for the purpose of constructing a Texture Profile of a dietary item, according to an embodiment of the invention as disclosed herein. The tags as disclosed in the diagram are merely illustrative, and are not meant to be exhaustive or to limit the scope of invention in any manner

FIG. 11 is a flowchart illustrating a system and method of constructing a Texture Profile (1100) of a dietary item, according to an embodiment of the invention as disclosed herein.

FIG. 12 is a schematic representation of generating ingredient profile and recipe profile of a dietary item in accordance with a preferred embodiment of the present invention.

FIG. 13 is a schematic representation illustrating the criteria for grouping (1300) of dietary items and dishes in accordance with a preferred embodiment of the present invention.

FIG. 14 is a schematic representation illustrating determination of the intensity score of a dietary item as per one of the preferred embodiments of the invention.

FIG. 15 is a flowchart illustrating a system and method of constructing a user signup process along with constructing an initial food preferences profile of the user, according to a preferred embodiment of the invention as disclosed herein.

FIG. 16 is a flowchart illustrating different sub-profiles in a final user food profile in accordance with a preferred embodiment of the invention as disclosed herein.

FIG. 17 is a flowchart illustrating a system and method of recommending cuisines, recipes and other necessary information based on the user requirements and preferences ranging from comfort to exploratory food habits, according to an embodiment as disclosed herein.

FIG. 18 is a flowchart illustrating a system and method of using user's external and internal factors to build users' food profile, according to a preferred embodiment of the present invention as disclosed herein.

FIG. 19 is a flowchart illustrating a method of using a human voice to understand and analyse human mood/emotions and map them to the profile of dietary items to generate recommendations, in accordance with a preferred embodiment of the present invention as disclosed herein.

FIG. 20 is a flowchart illustrating a schematic representation of the overall architecture of curating and recommending dietary items based on users' voice input, in accordance with a preferred embodiment of the present invention as disclosed herein.

FIG. 21 is a schematic structural diagram of a computer system suitable for implementing the embodiment of the present disclosure.

### DETAILED DESCRIPTION OF THE INVENTION AND THE DRAWINGS

The disclosure of the present invention is further described below in detail in combination with the accompanying drawings and the embodiments. It may be appreciated that the specific drawings and embodiments provided herein are merely for illustrating the relevant disclosure, rather than limiting the disclosure. In addition, it should also be noted that, for the ease of description and sake of brevity, only the parts related to the relevant disclosure are shown in the accompanying drawings. Descriptions of well-known components and processing techniques are omitted, so as not to unnecessarily obscure the embodiments herein. The examples used herein are intended merely to facilitate an understanding of ways in which the embodiments of the present invention can be practiced and to further enable those skilled in the art. It should be noted that the embodiments in the present disclosure and the features in the embodiments may be combined with each other on a non-conflict basis. Accordingly, the examples should not be construed as limiting the scope of the present invention.

Embodiments of the present invention may comprise or utilize a special purpose or general-purpose computer including computer hardware, such as, for example, one or more processors and system memory, as discussed below. Embodiments within the scope of the present invention also include physical and other computer-readable media for carrying or storing computer-executable instructions and/or data structures. By way of example, and not limitation, embodiments of the invention can comprise at least two distinctly different kinds of computer-readable media: computer storage media and transmission media. Computer storage media includes RAM, ROM, EEPROM, CD-ROM or other optical disk storage, magnetic disk storage or other magnetic storage devices, or any other medium. Computer implemented program may comprise one or more computer-readable storage media having encoded thereon computer-executable instructions which, when executed upon one or more computer processors, perform the methods, steps, and acts as may be described in the present invention.

In one of the most general embodiment of the present invention is provided a method of generating personalized dietary recommendation information for a user, which method is implemented or assisted by way of a computer or hardware devices or network thereof connected with each other through web or local area network or through any other means. Another embodiment of the present invention involves a system for generating personalized dietary recommendation information for a user, wherein the system comprising computer and hardware devices which are capable of executing the instructions for performing the method as disclosed in the present invention.

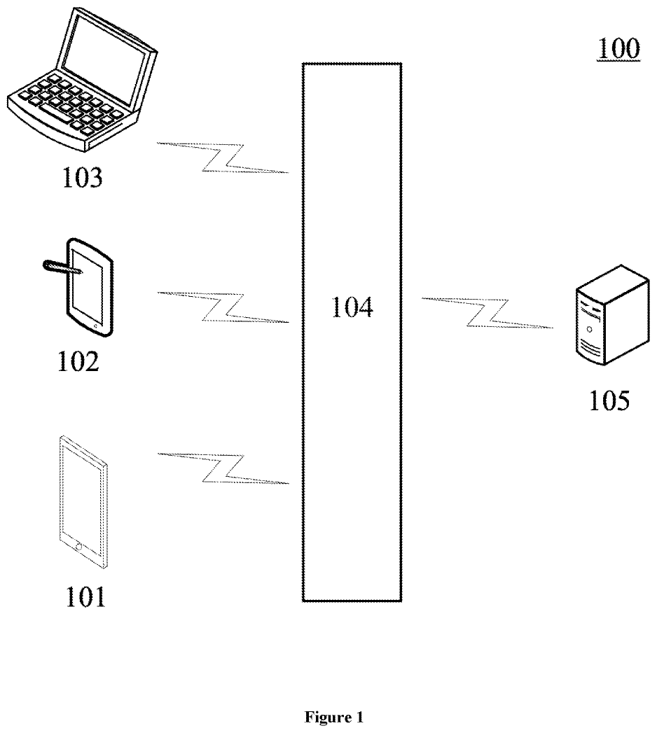

FIG. 1 illustrates a conceptual block diagram of system architecture 100 in which the present invention may operate. As shown in FIG. 1, the system architecture 100 may include terminal devices 101, 102, 103, a network 104, and a server 105. The network 104 is to provide a communication link medium between the terminal devices 101, 102, 103 and the server 105. The network 104 may include various types of connections, such as wired, wireless communication links, optic fibers, etc. A user may interact with the server 105 through the network 104 using the terminal devices 101, 102, 103. Various client applications may be installed on the terminal devices 101, 102, 103, such as web browser applications, search applications, profile applications, instant messaging tools, mailbox clients, social platform software, etc.

The terminal devices 101, 102, and 103 may be various electronic devices having display screens, including but not limited to smart phones, tablet computers, laptop computers, and desktop computers. The server 105 may be a server that provides various services, such as a background server that provides support for database or profile applications displayed on the terminal devices 101, 102, and 103. The background server may perform analysis and other processing on received data from the users, such as a preferences, and display back a processing result such as a recommendation of a dietary item, cuisine, location of a restaurant, etc. to the terminal devices.

It should be noted that the method for recommendation of dietary item or cuisine provided by the embodiments of the present disclosure is generally performed by the server 105, accordingly, the apparatus for outputting the recommendation is generally provided in the server 105. It should be understood that the number of terminal devices, networks, and servers in FIG. 1 is merely illustrative. Depending on the implementation needs, there may be any number of terminal devices, networks, and servers.

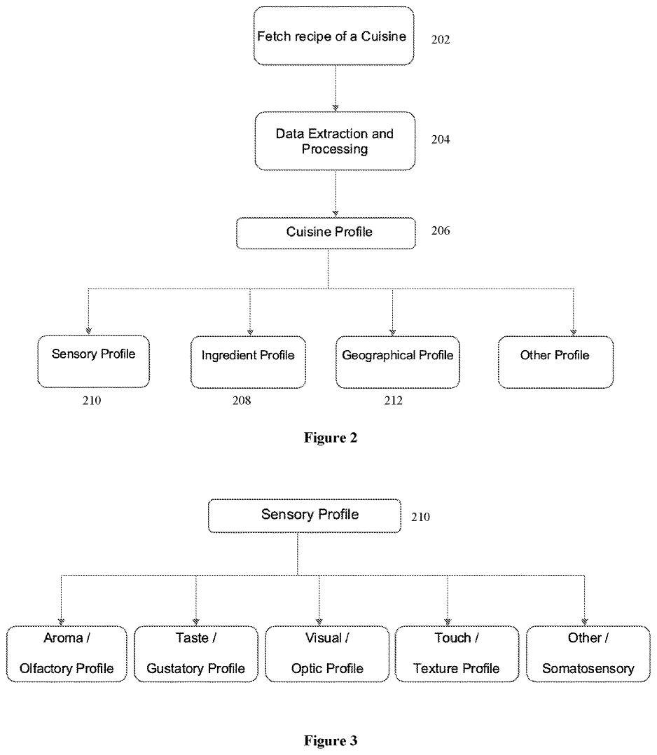

FIG. 2 is a diagrammatic representation illustrating the system and method of constructing an Ingredient Profile of a dietary item in accordance with a preferred embodiment of the present invention. According to the embodiment, a computer implemented device is configured to fetch (202) and process (204) the data of a dietary item or a cuisine. The processing of data is carried out to construct the Cuisine Profile (206) of the cuisine. The Cuisine Profile may typically comprise an Ingredients Profile (208), a Sensory Profile (210), and a Geographical Profile (212). There may be other profiles, such as recipe profile, etc.

FIG. 3 is a diagrammatic representation illustrating the constitution of a Sensory Profile of a dietary item in accordance with a preferred embodiment of the present invention. According to the embodiment, a computer implemented device is configured to create the Sensory Profile of the dietary items. Sensory profile of a cuisine may include all the information about the cuisine which stimulate any one or more of the five senses of a human being, and includes the following profiles:
Smell/Aroma/Olfactory Profile
Taste/Gustatory Profile
Visual/Optic Profile
Touch/Texture Profile
Sound/Auditory Profile
   These profiles are generated on the basis of many attributes and factors of the dietary item/cuisine, such as the quantity and quality of ingredients, method of cooking or processing, etc. The Sensory Profile, in one embodiment of the invention, also includes the Somatosensory Profile, which pertains to the secondary neural sensation in the brain upon consumption of the dietary item.

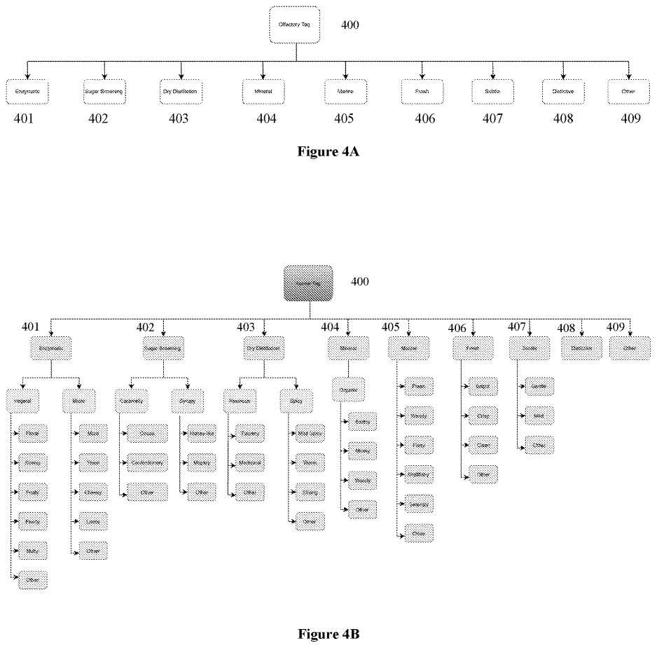

FIG. 4A and FIG. 4B are diagrammatic representations depicting different Olfactory/Aroma Tags (400) in an Olfactory/Aroma Profile of a dietary item, according to an embodiment of the invention as disclosed herein. Aroma/Olfactory Profile is an aggregate of all the aromatic indicators and tags (400) of a dietary item. Aroma tags are created according to one or more chemical properties of the ingredients. The chemical properties of the ingredients are mapped to extract the features by the ingredient combination and ingredients quantity in the recipe of the dietary item or cuisine. For instance, the tag ‘sugar browning’ (402) may be used for caramel custard depending upon the ingredient sugar and its method of processing of the ingredient in the cuisine recipe. There may be multiple tags associated with a cuisine, with different intensity scores associated to each tag. In an embodiment, the Aroma Profile provides complete information regarding different aromas derived either from a plant or animal in a cuisine. In one of the most preferred embodiment, the Aroma Profile also includes the score of aroma tags in the final dietary item/cuisine prepared after cooking/processing.

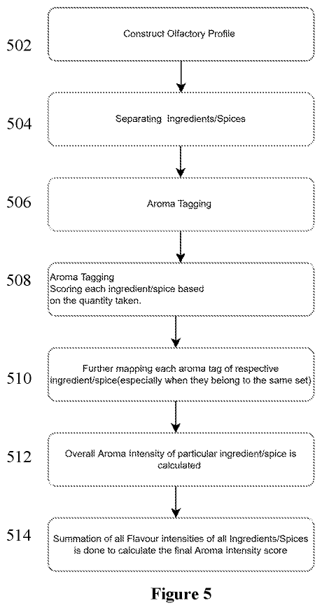

FIG. 5 is a flowchart representation illustrating a systematic method of constructing an Aroma/Olfactory Profile (502) of a dietary item, according to an embodiment of the invention as disclosed herein. According to the embodiments, a computer implemented device is configured to create the aroma profile of the dietary items. The Aroma profile is constructed by first separating the ingredients/spices (504) of a cuisine, and then carrying out the aroma tagging (506) of the separated ingredients/spices. During the aroma tagging process, a scoring (508) is provided to each ingredients/spices depending upon the quantity used in the cuisine recipe. After scoring, mapping (510) is carried out for each aroma tag with respect to the ingredients/spices, especially when the tags belong to the same set. Common categories arranged in a hierarchical manner are mapped to the respective aroma tags. Overall aroma intensity of particular ingredients/spices is calculated (512) and these overall aroma intensities of all ingredients/spices is added to calculate the final Aroma Intensity Score of the cuisine (514). The Aroma Intensity score is used as one of the indicators to determine a user's inclination for a dietary item based on its smell. Every user has an aromatic score range for mapping with the Aromatic Intensity Score of the dietary items or cuisines.

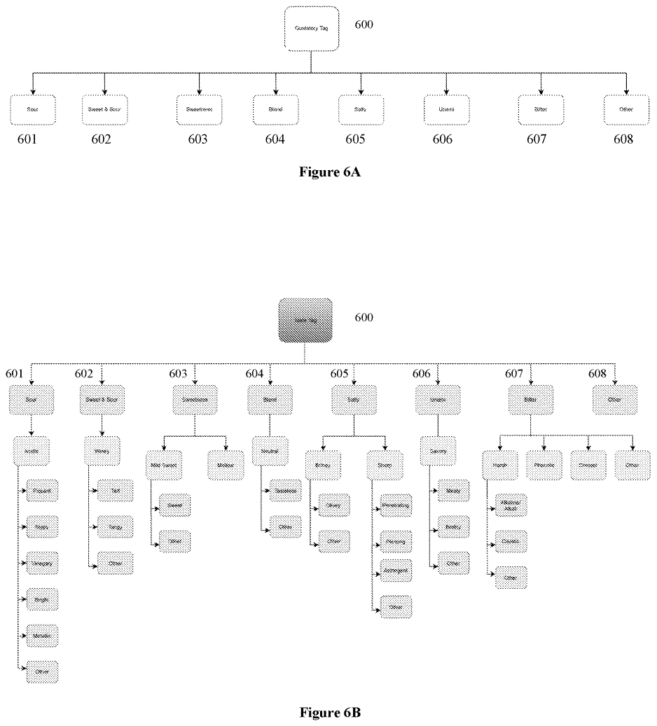

FIGS. 6A and 6B are diagrammatic representations depicting different Gustatory/Taste Tags (600) in the Gustatory/Taste Profile of a dietary item, according to an embodiment of the invention as disclosed herein. Gustatory/Taste Profile is an aggregate of all the taste indicators and tags (600) of a cuisine. Taste tags are created according to one or more chemical properties of the ingredients. The chemical properties of the ingredients are mapped to extract the features by the ingredient combination and ingredients quantity in the recipe of the cuisine. For instance, the tag ‘sweetness’ (603) may be used for a cake and the tag ‘salty’ (607) may be used with pasta, depending upon the ingredient and method of processing of the ingredient in the cuisine recipe. Further, as evident from the FIG. 6B, each tag may have further categorization into sub-tags for more specific tagging. There may be multiple tags associated with a cuisine, with different scores associated to each tag. In an embodiment, the Taste Profile provides complete information regarding primary tastes such as sweet, sour, salty, bitter, umami and others. In one of the most preferred embodiment, the Taste Profile also includes the score of taste tags in the final dietary item/cuisine prepared after cooking/processing. For instance, a dietary item which has sweet and sour/acidic flavours or ingredients will have a tangy flavour, and depending upon the method of preparation the intensity of tanginess may vary; accordingly, the taste profile of the dietary item will include the ‘tangy’ tag with the relevant intensity score.

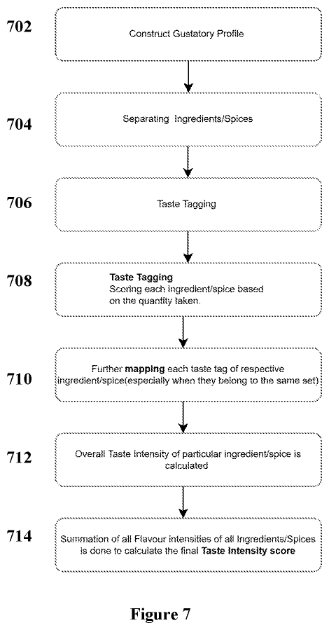

FIG. 7 is a flowchart representation illustrating a systematic method of constructing an Gustatory/Taste Profile (702), of a dietary item, according to an embodiment of the invention as disclosed herein. According to the embodiments, a computer implemented device is configured to create the taste profile of the dietary items. The taste profile is constructed by first separating the ingredients/spices (704) of a cuisine, and then carrying out the taste tagging (706) of the separated ingredients/spices. During the taste tagging process, a scoring (708) is provided to each ingredients/spices depending upon the quantity used in the cuisine recipe. After scoring, mapping (710) is carried out for each aroma tag with respect to the ingredients/spices, especially when the tags belong to the same set. Common categories arranged in a hierarchical manner are mapped to the respective taste tags. Overall taste intensity of particular ingredients/spices is calculated (712) and these overall aroma intensities of all ingredients/spices is added to calculate the final Taste Intensity Score of the cuisine (714). The Taste Intensity score is used as one of the indicators to determine a user's inclination for a dietary item based on its taste. Every user has a taste score range for mapping with the Taste Intensity Score of the dietary item. In one of the most preferred embodiment, the profile also includes taste intensity score of taste tags in the final dietary item/cuisine prepared after cooking/processing, including the mouthfeel taste tags and aftertaste tags.

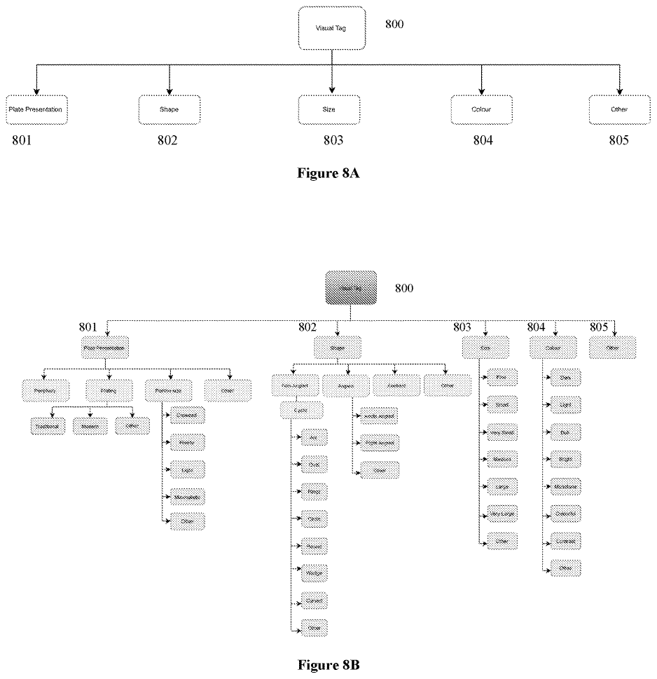

FIGS. 8A and 8B are a diagrammatic representation illustrating different Visual Tags (800) of Visual Profile, of a dietary item, according to an embodiment of the invention as disclosed herein. According to the embodiment, a computer implemented device is configured with pre-defined tags and also to create new tags to determine and define the visual appearance of a dietary item in the form of tags. The Visual profile is an aggregate of all the visual indicators of a dietary item stored in the language of Visual Tags (800). The tags are categorized based on various factors such as plate presentation, central object, peripheral object, colours, colour contrast, shapes, sizes, patterns, and surface textures, among others as depicted in the FIG. 8A. An illustrative, but non-exhaustive set of tags, for some of these categories are provided under FIG. 8B. For instance, under the category Colour (804), the Tags could be dark, light, dull, bright, monotone, contrast, etc. In accordance with a preferred embodiment of the invention, since the visual tags of the cooked/processed dietary item may be different from the visual tags of the ingredients, the visual profile includes the visual tags and the corresponding intensity scores of the visual tags.

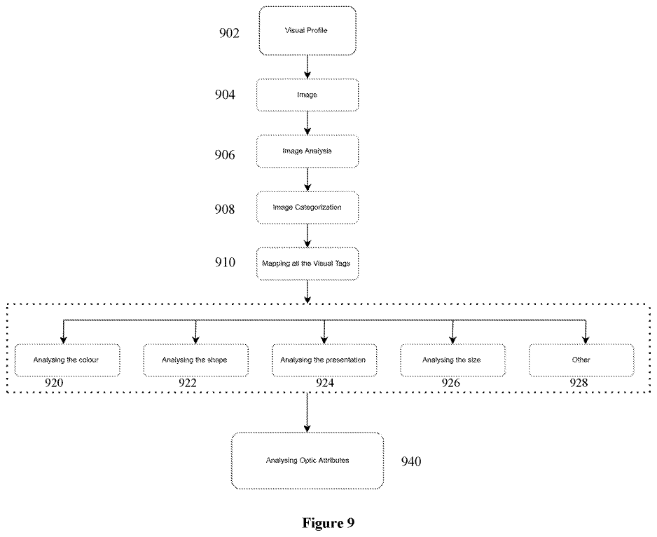

FIG. 9 is a flowchart illustrating a systematic method of constructing a Visual Profile (902) of a dietary item, according to an embodiment of the invention as disclosed herein. According to the embodiments, a computer implemented device is configured to create the Visual Profile (902) of the cuisine. The Visual Profile (902) is constructed by first obtaining the image of the dietary item/cuisine (904), and then carrying out the image analysis (906). Every image of the dietary item/cuisine undergoes image processing and analysis at every segment using an image processing unit. Once the image is analysed, the image categorization (908) is done to enable the mapping (910) of all visual tags with the analysed and categorized image. As per one of the embodiments, a statical feature extraction unit is configured to extract one or more statistical features from the images, and the extracted one or more statistical features are mapped (910) to the predefined visual tag set. During such mapping, different visual attributes of the dietary item/cuisine are analysed. For instance, the analysis of colour (920), shape (922), presentation (924), size (926), etc. In an embodiment, Visual Profile provide information regarding the presentation, shapes, sizes, colours, and other factors of the dietary item. The Visual attributes in the form of Visual Profile is used as one of the indicators to determine a user's inclination for a dietary item based on its visual appeal.

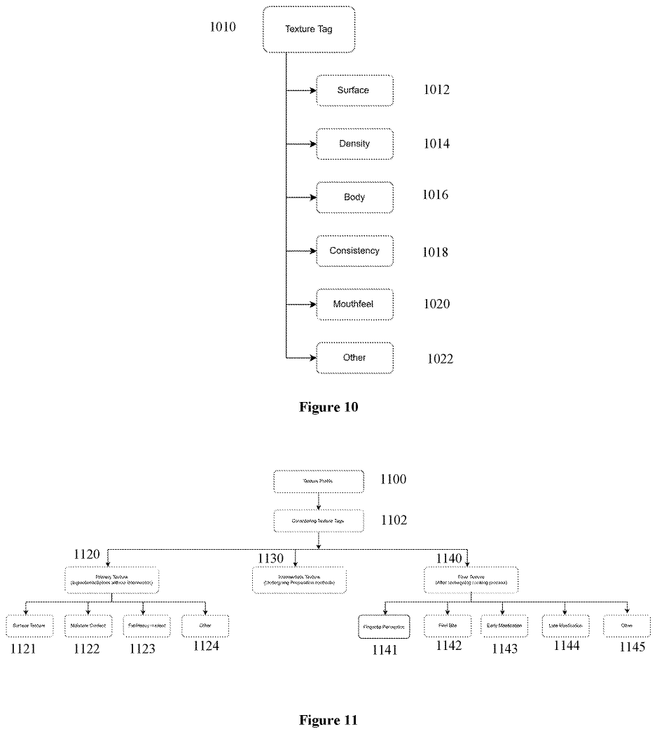

FIG. 10 is a diagrammatic representation illustrating different Texture Tags (1010) for the purpose of constructing a Texture Profile of a dietary item, according to an embodiment of the invention as disclosed herein. According to the embodiment, a computer implemented device is configured with pre-defined tags and also to create new tags to determine and define the texture of a cuisine in the form of tags. The Texture profile is an aggregate of all the texture indicators of a dietary item stored in the language of Texture Tags (1010). Some of the prominent categories of texture tags as illustrated are based on various factors such as surface of cuisine (1012), density of cuisine (1014), body (1016), consistency (1018), mouthfeel (1020), moisture content (1122), etc. Texture profile is an aggregate of all the textural indicators and tags of a cuisine.

FIG. 11 is a flowchart illustrating a system and method of constructing a Texture Profile (1100) of a dietary item, according to an embodiment of the invention as disclosed herein. According to the embodiments, a computer implemented device is configured to create the Texture Profile (1100) of a dietary item/cuisine. According to one of the embodiments, the Texture Profile (1100) is constructed by considering and analysing all the texture tags (1102) and determining the Primary Texture (1120), Intermediate Texture (1130) and Final Texture (11400) of the cuisine. The Primary Texture (1120) is deduced on the basis of the texture of raw ingredients/spices without any intervention. This is considered for ingredients in their natural state that have not undergone any intervention. Primary texture profile is constructed by analysing the surface textures, moisture content, cohesiveness, viscosity, fractur ability among other factors. For instance, the moisture content (1122) of the vegetable involved in the cuisine is taken into account and different texture tags may be attributed on the basis of whether it is dry, moist, watery, wet, etc. Likewise, there may be different tags under the fat/heavy content (1123) wherein the texture tag could be fatty, oily, greasy, etc. As per one embodiment, the Intermediate Texture (1130) takes into account the preparation method adopted in the cuisine recipe and is considered for the ingredients that have undergone preparation methods such as chopping, grinding, blending, etc. For instance, the different tags that may be allotted for the Intermediate Texture includes grated, cut, minced, sliced, chopped, shredded, cut, etc. These texture tags are useful in defining the method of preparation of the cuisine. The Final Texture (1140) is the texture after cooking/processing of the cuisine, and is considered for the ingredients that have undergone the process of cooking. The categories of texture tags for final texture may include the fingertip perception, first bite, early/late mastication, etc. In an embodiment, the term Texture tag provides complete information regarding description of surface, density, body, and mouthfeel, viscosities of liquid, semi-liquids, and cohesive factors of the dietary items which acts as indicators to determine a user's inclination for a cuisine. As per another preferred embodiment, the final texture of the cuisine undergoes processing using a processing unit and one or more statistical features extracted and mapped to the predefined texture tag set.

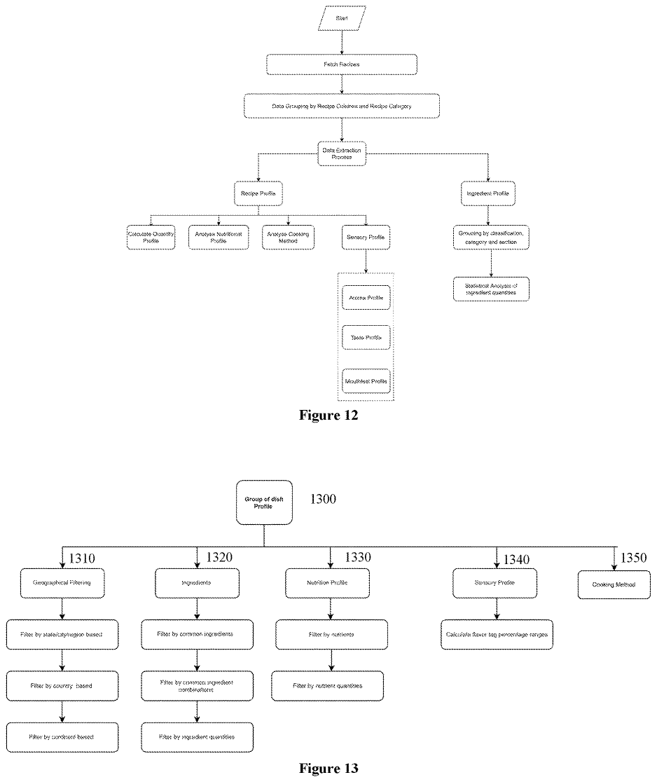

FIG. 12 is a schematic representation of generating ingredient profile and recipe profile of a dietary item in accordance with a preferred embodiment of the present invention. As per the embodiment, once the recipe is fetched, the data grouping is done on the basis of recipe. The recipe profile is constructed after analysis of the nutritional profile, cooking method, sensory profile, etc. Likewise, for the ingredients profile, first the grouping is done on the basis of classification category and then the statistical analysis of the ingredients quantities is carried out. The ingredient profile includes the information about the kind, quality, quantity, and nature of all the ingredients. Likewise, the sensory profile includes information about the aroma profile, taste profile, texture profile, visual profile of the dietary item as illustrated above.

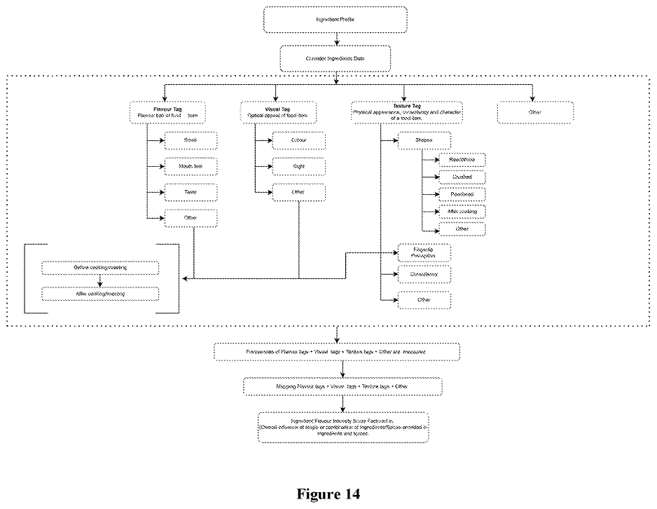

FIG. 13 is a schematic representation illustrating the criteria for grouping (1300) of dietary items and dishes in accordance with a preferred embodiment of the present invention. As per the embodiment, once a cuisine is profiled, it may be grouped as per the Geographical location (1310) wherein the filters can be placed on the basis of continent, region, country, city, or landscape of a country. Same dietary item may have different ingredients, style of cooking and textures, smell, taste depending upon the geographical location. Likewise, the grouping can be done on the basis of ingredients (1320), their quantities, or combinations thereof. For instance, the vegetarian or non-vegetarian, fish, kosher, halal, etc. Other categories of grouping could be based on nutritional profile (1330), sensory profile (1340) or cooking method (1350) of the dietary item. The dietary recommendations to the users can be made on the basis of these grouping mapped with the user's preferences and choices. Also, the dietary items are searchable by the user on the basis of these groupings.

FIG. 14 is a schematic representation illustrating determination of the intensity score of a dietary item as per one of the preferred embodiments of the invention. As per the embodiment, the ingredient data from the Ingredient Profile of a dietary item/cuisine is considered. On the basis of the ingredients, their quality, and quantity, the Sensory profiles are generated, wherein the frequencies of flavour tags are calculated. Once these tags are mapped, the Ingredient Flavour Intensity Scores are factored in. The Ingredient profile is the relative flavour intensity such as Aromas, tastes, mouthfeel, aftertastes, and textural attributes of an ingredient. In accordance with a preferred embodiment of the present invention, the Ingredient Profile is generated by first measuring frequencies of Visual Profile, Flavour Profile, Texture Profile, and other profiles. Thereafter, the common categories arranged in a hierarchical manner are mapped to the respective profiles and the patterns are observed. Intensity Scores are also factored in to derive the Ingredient Profile.

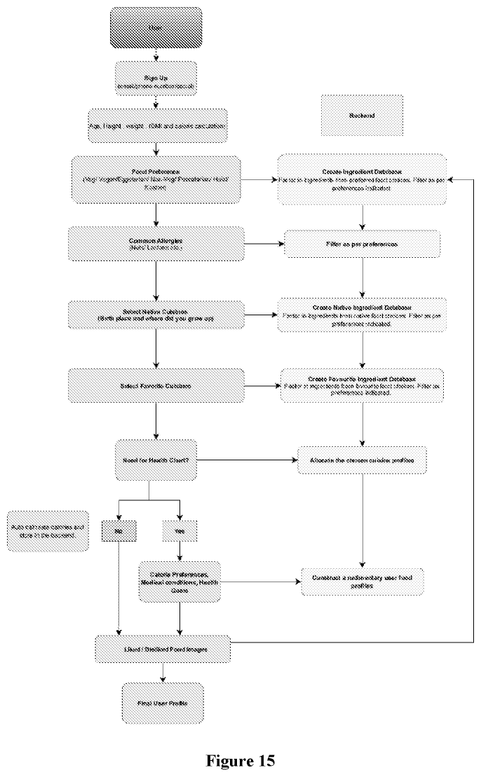

FIG. 15 is a flowchart illustrating a system and method of constructing a user signup process along with constructing an initial food preferences profile of the user, according to a preferred embodiment of the invention as disclosed herein. According to the embodiments, a computer implemented device is configured to create the user profile. The user signup process may involve acquiring the user's preference in terms of: (i) ingredients of the food, such as vegetarian, pescatarian, halal, kosher, and also the food allergies, if any, such as allergy to nuts, shellfish, gluten, etc.; (ii) comfort food, which is deducible from the native dietary items/cuisines; (iii) sensory attributes of the dietary items, which may deducible from the selection of native cuisines, acquired tastes, calorie preferences, health goals, medical conditions, etc. After which, as per an embodiment, the profile optimization is done to refine the food profile by letting the user to swipe (right for like or left for dislike) on dish images based on their preference. Based on the choices made by the user, a user profile is created. As per a preferred embodiment, user profile may also include user's cognitive behaviour and patterns such as moods, emotions, health goals, allergies, medical conditions, food habits, etc. As per another embodiment, the user profile also includes external factors such as user's current location, weather conditions, time of the day, occasion, space, ambience, etc. In another preferred embodiment, the user also include a score of user to explore and try new dietary items outside the comfort food range of the user. These additional attributes in the user profile aide in defining the range of confidence score while generating recommendation of the dietary items to the user.

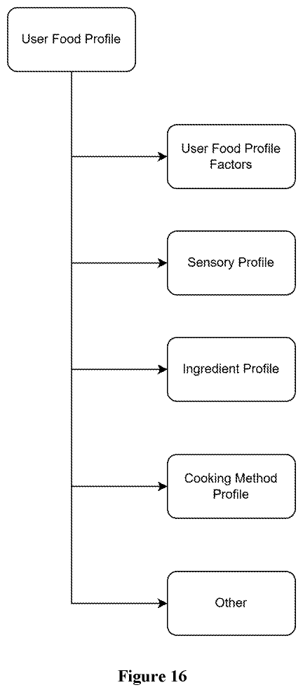

FIG. 16 is a flowchart illustrating different sub-profiles in a final user food profile in accordance with a preferred embodiment of the invention as disclosed herein. The method includes building the user food profile based on user profile factors, sensory profile, ingredient profile, cooking method profile and others.

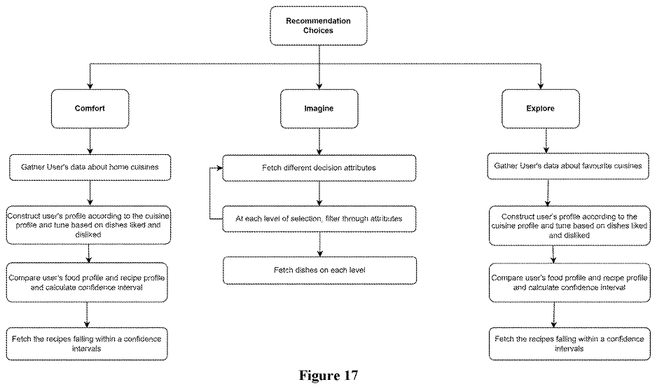

FIG. 17 is a flowchart illustrating a system and method of recommending cuisines, recipes and other necessary information based on the user requirements and preferences ranging from comfort to exploratory food habits, according to an embodiment as disclosed herein. The term dietary recommendation, in accordance with a preferred embodiment, involves recommending the dietary items, dishes, cuisines, beverages, etc. based on the user preferences namely comfort, crave/imagine and explore. In accordance with a preferred embodiment of the invention, the confidence intervals of the recommended dietary items in the comfort food have a narrower range. In another preferred embodiment, the imagine/crave category of recommendations have a wider interval than the comfort food, and the explore category has the widest. In another preferred embodiment, there could be multiple categories of the user's preference from comfort to explore.

In an embodiment, if the user preference is comfort, the data of user's comfort food and home cuisines is gathered first, and the dish recommendation is performed. User's food profile and recipe profile are compared to calculate the confidence intervals, and only the recipes falling within the confidence intervals are fetched and recommended. In an embodiment, if the user preference is imagine or craving, the dish recommendation is curated by designing a user crave profile by asking the user a list of questions to understand the users crave preferences. The crave profile is then optimized using users' ingredient profile factors and the users' sensory profile. Different decision attributes are fetched and at each level of selection different filters are applied. The dish recommendation is then curated based on the crave profile analysis. The choices which are offered to the user while creating the profile are selected in a manner so as to obtain maximum information about the user's preference in the least number of steps for selection. In an embodiment, if the user preference is exploring, the dish recommendation is performed after gathering User's favourite cuisines and user's food profile and recipe profile are compared to calculate the confidence intervals, and all the recipes falling within the confidence intervals for the explore category are fetched and recommended.

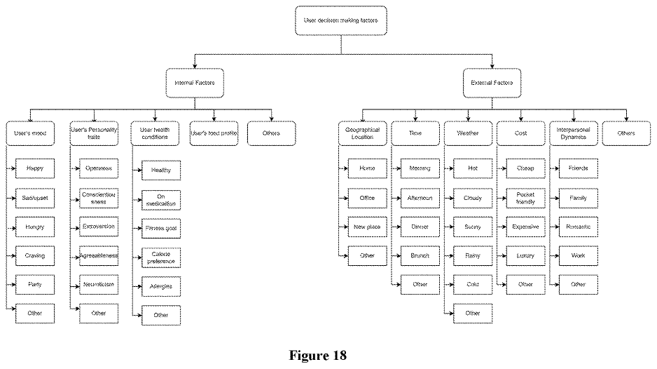

FIG. 18 is a flowchart illustrating a system and method of using user's external and internal factors to build users' food profile, according to a preferred embodiment of the present invention as disclosed herein. User profile factor is a combination of various factors that are analysed across each category as they influence the prediction model. The profile factors are classified into external triggers (such as time, space, interpersonal dynamics, weather, Distance, Price points, location, ambience, etc.) and internal triggers (such as behaviour, food habits, cognitive processes, moods & emotions, personal preferences, health goals, medical conditions, etc.)

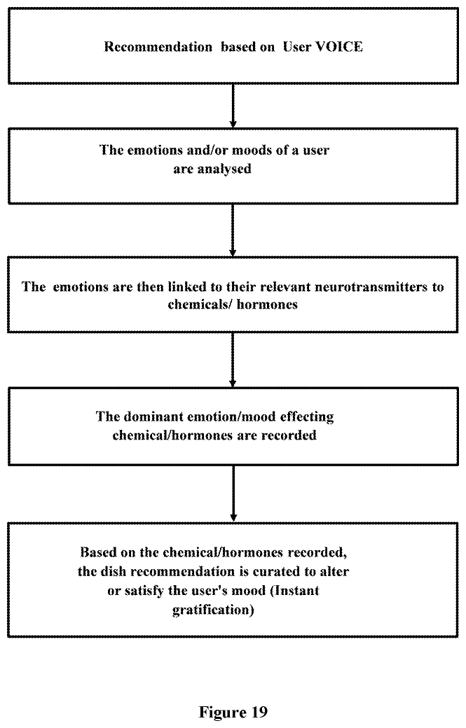

FIG. 19 is a flowchart illustrating a method of using a human voice to understand and analyse human mood/emotions and map them to the profile of dietary items to generate recommendations, in accordance with a preferred embodiment of the present invention as disclosed herein.

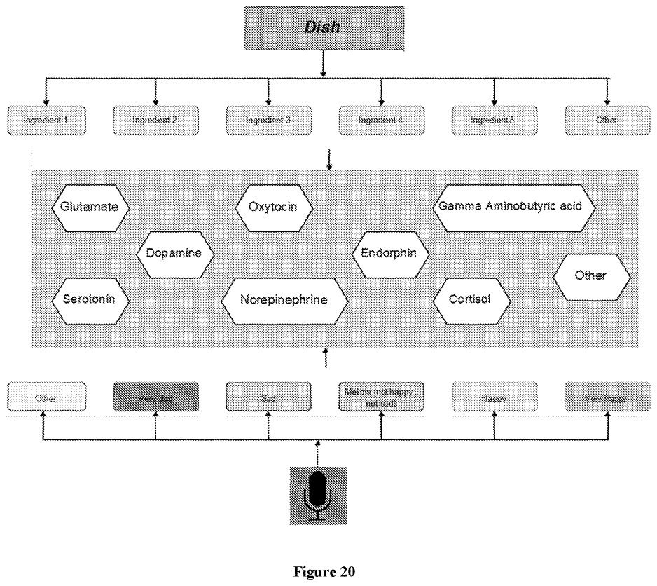

FIG. 20 is a flowchart illustrating a schematic representation of the overall architecture of curating and recommending dietary items based on users' voice input, in accordance with a preferred embodiment of the present invention as disclosed herein. According to a preferred embodiment of the invention, the voice-based food recommendation is performed based on the following:

Initially, the user's input voice, i.e., raw audio data is broken down using various categories, which may include:
i. The lexical features (the kind of vocabulary used).
ii. The acoustic features (voice sound properties like the tone, pitch, jitter, noise, speech rate etc.).
iii. Voice dimensions (energy, term, and adequacy of voice level proportions, etc).

The above categories are further analysed using parameters such as the speech rate, Pitch range, Pitch changes, Intensity, Voice quality, Articulation, etc. are mapped to one of the human emotions/moods. As per another embodiment, Robert Plutchik's wheel of emotions is considered to understand the fundamental emotions. As per another embodiment, these human emotions/moods are analysed and mapped to the neurotransmitters and the chemicals/hormones released in the brain, the dominant emotions/mood affecting chemicals/hormones are recorded. Finally, as per another embodiment, the chemicals/hormones recorded are stored and compared against dish ingredients chemical compositions. As per an embodiment, the dietary recommendation to the user takes into account the user's emotion/mood at the pertinent time after matching the chemicals/hormones with the relevant dietary items.

In another preferred embodiment of the invention, the user's amenability to try and experiment new dietary items outside the comfort range is also recorded in the form of a score which constitutes a part of the user profile. The score keep changing, depending upon the user's preferences and inputs. In one embodiment, this score is visible to the user, and can be adjusted by the user. The user's profile also gets curated with each preference of selection as well as rejection of dietary item recommendation exercised by the user.

As per another embodiment, the system and method includes building a food profile for the user, which is based on the user's native cuisines, user's acquired tastes over a period, foods that the user is allergic to, desired calorific range of the user, spice levels, medical conditions, keenness to experiment new food, etc. Since every choice that the user makes is a product of a cognitive processing patterns such as behavioural, visceral, or reflective processes, the system is configured to learn, customize and personalise the user profile with each additional preference or information collected from the user. In an embodiment, the user's profile constitutes common ingredients that are part of the user's frequent diet, sensorial profile (visual, aromatic, taste, texture, mouth-feel patterns) information and cooking methods.

In an embodiment, the user's profile is constructed, curated, modified, managed, stored, and presented by an automated computer implemented device enabled with the techniques of self-learning and artificial intelligence. In another embodiment, the computer implemented device is connected with the user through an internet protocol and an application interface, wherein the device is configured to receive the information from the user for constructing, curating, modifying, managing, storing, and presenting the user profile on a real-time basis. According to one of the general embodiment of the present invention, a personalized recommendation engine is built that factors the above-mentioned variables along with other categorized or uncategorized variables collected over a period to improve the effectiveness of the recommendation. The nature of the system is such that it evolves continuously by factoring in new variables fit for its effectiveness.

In an embodiment, the system and method includes profiling each dietary item by assigning a flavour complexity score. This process includes breaking down the dietary items at ingredient level and pre-assigning the ingredients with flavour tags. Further, the flavour complexity score is calculated based on a quantity, frequency, and variety of flavour tags of the ingredients. As per one embodiment, the profile of each dietary item includes individual nutritional characteristics, molecular ingredients, micronutrients, phytonutrients, macronutrients, chemicals, additives, antioxidants, and spices used in the dietary item. Additives to the dietary items may comprise preservatives, coloring agents, flavors, fillers, etc. In another embodiment, the profile also includes geolocation and availability information in different restaurants around the user.

In an embodiment, the profile of each dietary item is constructed, curated, modified, managed, stored, and presented by an automated computer implemented device enabled with the techniques of self-learning and artificial intelligence. The computer implemented device is enabled to decode a recipe or a dietary item into type and amount of known or unknown or unlisted ingredients. The device is also enabled to decrypt, identify and record the nutritional as well as sensory attributes of the dietary item. As per one embodiment, the computer implemented device is configured with trackers to fetch the information about recipes, ingredients, locations, restaurants, foods habits, nutritional profiles, etc. of dietary items from the existing databases on the web, and process the information to be stored in the format required under the present invention. As per another embodiment, the information about recipes, ingredients, locations, restaurants, foods habits, nutritional profiles, etc. is received and processed by the computer implemented device on a real time basis to update the database for mapping with the profiles of existing users.

In a preferred embodiment of the invention, the method includes analogizing and mapping the user profile against the dietary profiles already recorded in the database or system. The profiles are mapped to construct the confidence score for each of the mapped dietary items. A set of dietary items is identified wherein the dietary items have confidence score within a range. Recommendation information is generated for the user wherein the recommendation information includes at least one dietary item selected from the set of dietary items having the confidence score within the range. The range of the confidence score for recommendation may vary for each user, and also varies with changes in the profile of the user. In a most preferred embodiment, the range is calibrated with multitude of factors in the user profile. For instance, other than user's preference for ingredients, sensory attributes, and native food, the range may also vary depending upon the user's cognitive behaviour and patter (such as such as moods, emotions, health goals, allergies, medical conditions, food habits, etc.) as well as external factors (user's current location, weather conditions, time of the day, occasion, space, ambience, etc.). In an embodiment, the food recommended to the user falls within a confidence level (for example, 95%) of the user as a comfort food. The confidence interval may be larger (for instance 80%) when a user depicts personality traits such as openness to experience, adventurism, and generally a higher appetite for experimentation.

In another embodiment, only a selected dietary items having a confidence score within the range (say above 95%) are selected to be recommended to the user. The selection of the dietary items (from the set of dietary items meeting the criteria of having the confidence score within the range) is also carried out on the basis of the user profile and its attributes such as external factors, behavioural and cognitive patterns, etc.

In another embodiment, the system learns, identifies, and addresses the user's food needs that align with their behavioural and sensory inclinations. The system recommends food based on data provided by the user and a personalized sensory profile generated for each user.

In yet another embodiment, the system and method addresses the problem of indecisiveness related to food choices.

In yet another embodiment, wherein food attributes defined at a very high level of specificity provides the user with a presumptive sensory experience prior to tasting.

In yet another embodiment, the system will interface/integrate in real time with technologies including Internet of Things (IoT), Augmented Reality (AR), Virtual Reality (VR), Personal Digital Assistants, Robots etc.

In yet another embodiment, the system enables users to click a picture or upload a restaurant's menu. Further, it analyses the dishes, ingredients and/or additional content on the menu to filter dishes based on the user's food profile. Further, it tags dishes that can cause physical, physiological, emotional discomfort, thus indicating the prospective undesirable experience it may entail to the users. Further, it may add tips or suggestions allowing the users to communicate with the management or the chef for any modifications for a better experience.

In yet another embodiment, an AI driven dietary recommendation engine enables the user to add their frequently consumed or favourite food recipes. Further, it analyses the ingredients and sensory profiles of the added recipes. Further, it calibrates the added input to tailor any recipe suggestions it may provide to enhance the user experience.

In yet another embodiment, the system not only personalizes food recommendations for a user but also other ancillary offerings such as meal plans, diet plans, recipe books, marketplace, etc.

In an embodiment, the recommended dietary options are based on external factors such as time, weather and location. In an embodiment, the system and method enables food suggestions based on the user's emotions. In an embodiment, the method includes enabling the user a presumptive sensory description prior to trying a dish.

In an embodiment, the method includes enabling the user to add recipes to the food recommendation system, wherein the user is enabled to input data just by tap and adjust mechanism.

In an embodiment, the method includes enabling the user to share their day-to-day updates, recipe posts and related information on their social feed page for their friends and followers.

In an embodiment, the method enables the users to see their sensory match percentage, just by looking at the dishes or any dietary items.

In an embodiment, the system and method enables users to search by drawing patterns, by connecting bubbles (A, B, C, D, E etc.) on the screen. Each bubble represents a different variable, while entailing multiple categories under those variables (A1, A2, A3, etc.). The variables could be different ingredients, geographical locations, sensory attributes, etc. The user can select their preferred options (A3, B1, B2, C2, D1, D3, E5 etc.) and draw a pattern by connecting the bubbles across the screen. Different combinations yield different results. In an embodiment, the system and method enables users to select a variety of bubbles under different categories of variables. Here the user can pick and choose, add, or replace their choice of bubbles at any time. The variables may not be limited to food, but can also include any categories such as restaurants, places, goods and services, or anything that can be subjected to classification.

In an embodiment, the method enables the users to get an opportunity to dine with another user with a similar food profile for a limited time using virtual dining space. The virtual dining place would have different themes and environments one can choose from. Every day, the user will have an opportunity to meet a new person who shares similar tastes in food.

In an embodiment, the method includes combining more than one or more user to derive combined probabilities, and to recommend dietary items that fit both individual and/or group dietary profiles. For a group dietary recommendation, a group dietary profile is constructed wherein the data of one or more dietary items consumed by each user in the group of users is taken into account to create the group profile.

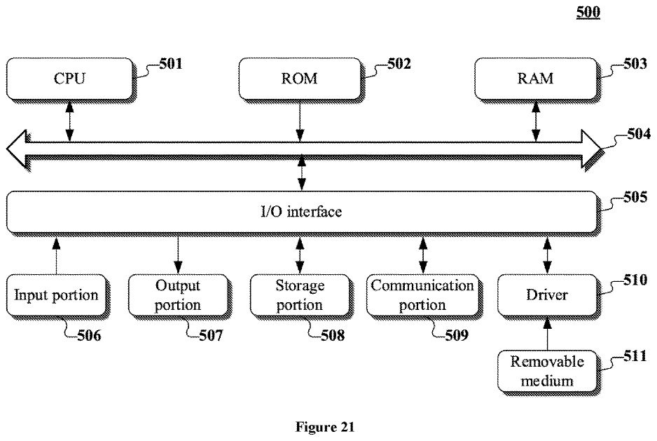

FIG. 21 shows a schematic structural diagram of a computer system 500 suitable for implementing an electronic device of an embodiment of the present disclosure. The electronic device shown in FIG. 21 is merely an example and should not impose any limitations on the functionality and scope of use of the embodiment of the present disclosure. As shown in FIG. 21, the computer system 500 includes a central processing unit (CPU) 501, which may execute various appropriate actions and processes in accordance with a program stored in a read-only memory (ROM) 502 or a program loaded into a random access memory (RAM) 503 from a storage portion 508. The RAM 503 also stores various programs and data required by operations of the system 500. The CPU 701, the ROM 502 and the RAM 503 are connected to each other through a bus 504. An input/output (I/O) interface 505 is also connected to the bus 504. The following components are connected to the I/O interface 505: an input portion 506 including, for example, a keyboard, and a mouse; an output portion 507 including, for example, a cathode-ray tube (CRT) and a liquid crystal display (LCD), and a speaker; a storage portion 508 including, for example, a hard disk; and a communication portion 509 including a network interface card such as a LAN card, a modem, or the like. The communication portion 509 performs communication processing via a network such as the Internet. The driver 510 is also connected to the I/O interface 505 as needed. A removable medium 511, such as a magnetic disk, an optical disk, a magneto-optical disk, a semiconductor memory, or the like, is installed on the drive 510 as necessary, so that a computer program read out therefrom is installed on the storage portion 508 as needed.

### Example

An example is provided below for the illustrations purposes only. As per a preferred embodiment:
n=total number of dietary items in the database
x=total number of dietary items extracted from ‘n’ set, based on users dietary preference and potential allergies after meeting the confidence score.
k=dietary items liked by the user within the ‘x’ set
i=a dietary items from ‘k’ set.
s=score derived by calculating the comparison of vectors of ‘i’ and ‘x’ sets
h=discarded dietary items containing potential allergens+dietary items containing ingredients restricted due to user dietary preferences within top 100 dishes (0≤h≤100)
a=100−h

Recommendation model: The user data is sent to the model, wherein the model extracts ‘x’ from ‘n’ after mapping the user profile with the profile of dietary items and identifying the confidence score. Then the cosine similarity is computed on these ‘x’ dishes against the ‘i’ (0≤i≤n). The vector of ‘x’ is compared with dietary items ‘i’ and the score ‘s’ is calculated. All the dietary items from set ‘k’ are subjected to the same process and their respective scores are calculated. As the process is repeated, the calculated scores (s_1, s_2, s_3 . . . s_k) are compared. Based on these scores, top 100 dishes are chosen.

In a preferred embodiment, the model is built on 3 levels of comprehensive analysis by machine learning algorithms. Level 1 (L1) analysis ensures the set ‘x’ is aligned with the user's dietary preferences (Vegetarian, Vegan, Eggetarian, Pescaterian, Halal etc.). The next step in the same L1 involves identification and elimination of dishes that contain ingredients with potential allergens which are recorded during the user's onboarding process from the top 100 dishes. Subsequently those dishes are discarded from the recommendation set. A score of 0.33 is given to set ‘a’ dishes and are passed to the Level 2 (L2) analysis.

Level 2 (L2) analysis is performed to compute sensory profile. Every ingredient is assigned sensory tags which are qualitative and quantitative by nature. Sensory tags may consist of aroma, taste, mouthfeel, texture & visual tags. Aroma, taste & mouthfeel are qualitative in nature where each of the sensory tags are given a score ranging from 0 to 1, where 1 indicates higher intensities and 0 indicates low intensities. The intensities are directly proportional to the quantity of the ingredient added. From set ‘k’, all the sensory tags are computed and their respective ranges are calculated in percentage. The computed tags and their calculated ranges are directly compared from set ‘a’. A score of 0.33 is assigned if each of the aroma, taste, mouthfeel, texture & visual tag fall within the range derived from set ‘k’. With these updated total scores (out of 0.66), dishes are pushed to Level 3 (L3) analysis.

Level 3 (L3) analysis is performed to group all the dishes into different clusters based on similarities between ingredients and other sensory attributes. User's native, favourite and cuisines derived from set ‘k’ will act as point of reference for identification of different clusters within the data set ‘a’. Recommendations will be given to the users from the aforementioned clusters and if a dish ‘i’ belongs to these clusters, an additional score of 0.33 will be attributed to its final score. The final recommendations are pushed forward which contains dish information along their probability scores.

### User Inputs:

Main Category: Non-Vegetarian
Sub Category: Halal
Native Cuisine: South-East Asia
Liked Cuisine: Indian
Allergy: Egg

### User Swipes on Homepage:

Liked Dishes: Chicken Biryani and Chicken Karahi

### Step by Step Working of Recommendation Model:

### Base Level:

1) Set ‘x’: Filtered dataset contains dishes that are non-vegetarian, halal categories and allergy (egg).
2) Set ‘k’: Chicken Biryani and Chicken Karahi
3) For each of the obtained set ‘x’ dishes, cosine similarity will be calculated with set ‘k’.
4) Top 100 dishes based on their cosine similarities will be obtained.

### Level 1:

1) Each of the recommended top 100 dishes will be broken down into ingredients.
2) Dishes are discarded if they contain potential allergens (egg) or if they contain ingredients restricted due to user dietary preferences (Non-vegetarian, Halal)
3) Score assigned to dishes from set ‘a’: 0.33.

### Level 2:

S1: score of tag with each dish
S2: user score of the S1 tag

$$d = absolute \vert s 1 - s 2 \vert$$

$$y = ( 1 - d ) * 0.01$$

1) Based on the set ‘k’, the user's sensory profile will be calculated.
2) For dishes from set ‘a’,
   compute S1 and S2.
   Calculate d and y
   the less the value of d, more be the value of y (0≤y≤0.066)
3) Step 2 is repeated for all dishes and all tags.
4) Scores will be added to dishes obtained from level 1. In the present case, “Veg Dum Biryani” will be having a 0.54 score, and “Shahi Paneer” will be having a 0.49 score.

### Level 3:

1) The cluster(s) based on native and liked cuisine will be obtained, in our case, one of the clusters will be: India, Pakistan, Bangladesh, and Afghanistan.
2) Since both of the recommended dishes belong to this cluster, a score of 0.33 will be added. Accordingly, the final probability scores for “Veg Dum Biryani” will be 0.87 (87%) and the score of “Shahi Paneer” will be 0.82 (82%).

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

1. A computer implemented method for generating custom dietary recommendation information for a user, the method comprising:
a. Compiling a database of plurality of dietary items;
b. Constructing a profile for each of the dietary item in the database, wherein the profile of each of the dietary item comprises:
(i) an ingredient profile of the dietary item;
(ii) a sensory profile of the dietary item, wherein the sensory profile comprises:
a visual profile comprising visual tags depicting visual attributes of the dietary item;
a taste profile comprising taste tags depicting taste attributes of the dietary item;
an aroma profile comprising aroma tags depicting aroma attributes of the dietary item;
a texture profile comprising texture tags depicting texture attributes of the dietary item;
(iii) A geographical profile of the dietary item, identifying at least one geographical territory associated with the dietary item;
c. Acquiring information and preferences from the user to construct user profile wherein the user profile comprises at least: one preferred ingredient or one preferred dietary item or one preferred geographical location;
d. Mapping the user profile against the profile of a plurality of dietary items in the database;
e. Constructing a confidence score for each of the mapped dietary items;
f. Creating a set of dietary items having confidence score within a range, wherein the said range is calibrated with the user's profile;
g. Generating recommendation information for the user wherein the recommendation information comprises at least one dietary item selected from the set of dietary items having the confidence score within the range; and
h. Presenting the recommendation information to the user.

2. The method as claimed in claim 1 wherein the said dietary items in the database includes cuisines, fruits, vegetables, ingredients, beverages, meat, edible plants or extracts thereof, etc.

3. The method as claimed in claim 1 wherein the said ingredient profile comprises the information about the kind, quantity, and quality of the ingredients in the dietary item including the information about the molecular ingredients, micronutrients, phytonutrients, macronutrients, chemicals, additives, antioxidants, and spices used in the dietary item.

4. The method as claimed in claim 1 wherein the said visual profile, taste profile, aroma profile, and texture profile comprises the intensity scores associated with the visual tags, taste tags, aroma tags, and texture tags respectively of the dietary item.

5. The method as claimed in claim 4 wherein the said intensity scores are in the form of range of values.

6. The method as claimed in claim 1 wherein the said profile of each of the dietary item further comprises information about the recipe for preparation of the dietary item including the information about cooking, roasting, processing, chopping, mixing, grinding, serving, etc. of all the ingredients.

7. The method as claimed in claim 1 wherein the said taste profile comprises tags and corresponding intensity score for the mouthfeel experience of the prepared dietary item after the requisite cooking, roasting, processing, chopping, mixing, grinding, etc. of the ingredients as per a recipe.

8. The method as claimed in claim 1 wherein the said sensory profile further comprises a somatosensory profile and an auditory profile of the dietary item.

9. The method as claimed in claim 1 wherein the said geographical location in the profile of dietary item is in the form of name of a city, district, state, country, continent, restaurant, hotel, outlet, etc. preferably the location which the dietary item is considered to be native to or is most commonly associated with.

10. The method as claimed in claim 1 wherein the said profile of each dietary item is constructed, curated, modified, managed, stored, processed, and presented by an automated computer implemented device enabled with the techniques of self-learning and artificial intelligence.

11. The method as claimed in claim 10 wherein the said automated computer implemented device is connected with the web to through an internet protocol, wherein the device is configured to fetch the information about the dietary items from the existing databases to construct profiles on a real-time basis.

12. The method as claimed in claim 1 wherein the said user profile includes information about the external factors such as user's current location, weather conditions, time of the day, occasion, space, ambience, etc.

13. The method as claimed in claim 1 wherein the said user profile includes information gathered from user's cognitive behaviour and patterns such as emotions, moods, health goals, allergies, medical conditions, food habits, etc.

14. The method as claimed in claim 1 wherein the said user profile is updated each time the user exercises and expresses a choice with respect to a dietary item whether recommended or otherwise.

15. The method as claimed in claim 1 wherein the user profile also includes a score depicting amenability of the user to experiment and try the dietary items outside the comfort range for the user, and wherein the said score is visible to the user and can be adjusted by the user.

16. The method as claimed in claim 1 wherein the said acquisition of information and preferences from the user to construct the user profile is carried out through an application interface on an electronic device, preferably a mobile/handheld device connected with the internet and configured with an application to display and receive information from the user on a real-time basis.

17. The method as claimed in claim 1 wherein the said user profile is constructed, curated, modified, managed, stored, and presented by an automated computer implemented device enabled with the techniques of self-learning and artificial intelligence.

18. The method as claimed in claim 17 wherein the said automated computer implemented device is connected with the user through an internet protocol and an application interface, wherein the device is configured to receive the information from the user for constructing, curating, modifying, managing, storing, and presenting the user profile on a real-time basis.

19. The method as claimed in claim 1 wherein the said mapping of the user profile against the profile of a plurality of dietary items is carried out by an automated computer implemented device configured with the techniques of self-learning and artificial intelligence wherein the said mapping, construction of confidence scores, and selection of dietary items having confidence score within a range is carried out on a real-time basis.

20. The method as claimed in claim 1 wherein the dietary recommendation information includes name, description, characteristics, ingredients, profile, image, recipe, or nutritional value of one or more dietary items. or a restaurant, geographical location, or a dietary plan, etc.

21. The method as claimed in claim 1 wherein the dietary items included in the said recommendation information are to enhance the sensory experience of the user.

22. The method as claimed in claim 1 wherein the said recommendation information is displayed to the user through an interactive application interface on an electronic device, preferably a mobile/handheld device connected with the internet.

23. The method as claimed in claim 1 wherein the said database is searchable by the user on the parameters of ingredients, taste, aroma, texture, visuals, geographical region, recipe, mood, emotions, occasion, nutritional value, allergies, restaurant, cooking method, etc.

24. The method as claimed in claim 1 wherein the said user constitutes a plurality of individuals such that the dietary recommendation generated is for the individuals to dine together.

25. The method as claimed in claim 1 wherein the said confidence score is visible to the user to assist in making a decision.

26. A system comprising:
one or more computer-readable memory devices storing data and instructions;
one or more input-output devices with graphic user interfaces;
one or more data processing apparatuses configured to interact with one or more memory devices and input-output devices, wherein upon execution of the instructions stored in the memory devices the system perform operations including:
a. Compiling a database of plurality of dietary items and storing it in one or more computer-readable memory devices;
b. Constructing a profile for each of the dietary item in the database, wherein the profile of each of the dietary item comprises:
(i) an ingredient profile of the dietary item;
(ii) a sensory profile of the dietary item, wherein the sensory profile comprises:
a visual profile comprising visual tags depicting visual attributes of the dietary item;
a taste profile comprising taste tags depicting taste attributes of the dietary item;
an aroma profile comprising aroma tags depicting aroma attributes of the dietary item;
a texture profile comprising texture tags depicting texture attributes of the dietary item;
(iii) A geographical profile of the dietary item, identifying at least one geographical territory associated with the dietary item;
c. Acquiring, through an input-output device with a graphic user interface, information and preferences from the user to construct user profile wherein the user profile comprises at least: one preferred ingredient or one preferred dietary item or one preferred geographical location;
d. Mapping the user profile against the profile of a plurality of dietary items in the database by the processing apparatus;
e. Constructing a confidence score for each of the mapped dietary items;
f. Creating a set of dietary items having confidence score within a range, wherein the said range is calibrated with the user's profile;
g. Generating recommendation information for the user wherein the recommendation information comprises at least one dietary item selected from the set of dietary items having the confidence score within the range; and
h. Presenting the recommendation information to the user through the input-output device with a graphic user interface.

27. A system as claimed in claim 26 wherein the said one or more computer-readable memory devices and processing apparatus are configured to store and process the techniques of self-learning and artificial intelligence.

28. A system for generating custom dietary recommendation information for a user, the system comprising:
a. a computer readable storage device storing a database of a plurality of dietary items;
b. a device configured to generate and store profile of each dietary items in the database, wherein the profile of each dietary item comprises:
i. an ingredient profile of the dietary item;
ii. a sensory profile of the dietary item, wherein the sensory profile comprises a visual profile comprising visual tags depicting visual attributes of the dietary item; a taste profile comprising taste tags depicting taste attributes of the dietary item; an aroma profile comprising aroma tags depicting aroma attributes of the dietary item; a texture profile comprising texture tags depicting texture attributes of the dietary item; and
iii. A geographical profile of the dietary item, identifying at least one geographical territory associated with the dietary item;
c. a graphic user interface (GUI) configured to interact with the user to receive the user's preferences and shared recommendations;
d. a device configured to generate profile of the user based on the preferences shared by the user, wherein the use profile comprises at least: one preferred ingredient or one preferred dietary item or one preferred geographical location;
e. a mapping device configured to map the user profile against the profile of a plurality of dietary items in the database; and
f. a processor device coupled with the mapping device to constructing a confidence score for each of the mapped dietary items and to segregate the information of dietary items having confidence score within a range, wherein the said range is calibrated with the user's profile;
g. a device to generate recommendation information for the user wherein the recommendation information comprises at least one dietary item selected from the set of dietary items having the confidence score within the range; and
h. an input/output device to transmit the generated recommendation to user's graphic user interface and to receive the user's response for analysis.
